(19) United States
(12) Patent Application Publication
(10) Pub. No.: US 2004/0197352 A1
(43) Pub. Date: Oct. 7, 2004

(54) Methods of improving or augmenting kidney function

(76) Inventor: Natarajan Ranganathan, Broomall, PA (US)

Correspondence Address: Jane Massey Licata, Licata & Tyrrell P.C., 66 E. Main Street, Marlton, NJ, 08053, US

(21) Appl. No.: 10/803,211

(22) Filed: Mar. 18, 2004

Related U.S. Application Data
(63) Continuation-in-part of application No. 10/689,359, filed on Oct. 20, 2003.
Application No. 10/689,359 is a continuation-in-part of application No. 09/855,346, filed on May 15, 2001, now Pat. No. 6,706,287.
Application No. 09/855,346 is a continuation-in-part of application No. 09/557,011, filed on Apr. 20, 2000, now Pat. No. 6,706,263.
(60) Provisional application No. 60/131,774, filed on Apr. 30, 1999.

(51) Int. Cl. C12N 1/20

(52) U.S. Cl. 424/234.1; 424/400; 435/252.5

(57) ABSTRACT

The present invention provides compositions for consumption by both animals and humans for the maintenance of a healthy gastrointestinal tract, the probiotic composition is efficacious in removing toxic nitrogenous by-products caused by chemotherapy and dietary imbalances such as high protein diets. The compositions of the present invention are also useful for delaying the onset of illness in patients with metabolic syndromes such as borderline diabetics or hypertension.

### CROSS REFERENCE TO RELATED APPLICATIONS

This application is a continuation-in-part of U.S. patent application Ser. No. 10/689,359 filed Oct. 20, 2003, which is a continuation-in-part of U.S. patent application Ser. No. 09/855,346 filed May 15, 2001, now issued as U.S. Pat. No. 6,706,287, which is a continuation-in-part of U.S. patent application Ser. No. 09/557,011 filed Apr. 20, 2000, now issued as U.S. Pat. No. 6,706,263, which claims the benefit of priority of U.S. Provisional Application 60/131,774 filed Apr. 30, 1999.

### GOVERNMENT SUPPORT

This work has been partly funded from the Small Business Innovation Research (SBIR) grant from the National Institutes of Diabetes, Digestive and Kidney (NIDDK) Diseases, National Institute of Health (NIH), Bethesda, Md., Grant No: 1R44DK61873-01.

### FIELD OF THE INVENTION

The present invention relates to compositions which provide probiotic bacteria to the gastrointestinal tract of a subject. The present invention is particularly desirable for human and veterinarian use to enhance kidney function by reducing the toxin load on the kidneys. The present invention is useful to remove toxic metabolites from the bowel of subjects with increased toxin loads, such as cancer or HIV patients undergoing chemotherapy, subjects with metabolic syndromes; and subjects consuming high protein and low carbohydrate diets.

### BACKGROUND OF THE INVENTION

Uremic toxins accumulate during development of renal failure. Uremic toxins are generated from a number of sources including diet, chemotherapy, diabetes and metabolic disorders. Any number of low-carbohydrate and high-protein diets are being touted today as the answer for weight problems and obesity. More and more people are cutting carbohydrates from their diets and eating more meat, fish, poultry and dairy products to reduce body fat. However, too much protein can lead to kidney problems and urinary tract problems, affecting a person's ability to stay properly hydrated during times of increased activity which also increases the risk of heat stress. Additionally, high-protein diets actually accelerate calcium loss from bones and increase the risk of osteoporosis, leading to an increase in stress fractures, ankle fractures and vertebral fractures. High protein diets also lead to the condition called ketosis, or the accumulation of ketone bodies.

Humans are not the only subjects affected by high protein diets. Other animals including pigs, horses, dogs and cats suffer from poor kidney and urinary tract functioning due to high protein diets. The gastrointestinal tract harbors a complex microbial ecosystem containing a large number and variety of bacteria. The resident bacterial population in the human gastrointestinal tract has a major impact on gastrointestinal function and thereby on health and well being. Among these, some bacteria are considered “probiotic”, in that they perform beneficial functions for the human organism (Holzapfel W H, et al. Int J Food Microbiol May 26, 1998; 41(2): 85-101).

Probiotic bacteria are known to stimulate the immune system and exert a competitive exclusion of pathogenic and putrefactive bacteria, reduce the amounts of ammonia and cholesterol in the blood, and promote absorption of minerals (von Wright, et al. Eur J Gastroenterol Hepatol November 1999; 11(11): 1195-1198). Additionally, probiotic bacteria produce antagonist effects against pathogenic microorganisms; stimulate the immune system; improve lactose digestion; are lypolytic thereby allowing fats to be more digestible; reduce plasma cholesterol; protect the intestinal mucosa, thereby assuring effective assimilation of the nutritive substances; produce polysaccharides that are active on some tumors; and reduce viability of some enzyme-producing microorganisms which catalyze the conversion of procarcinogenic substances into carcinogenic substances.

It is believed that the probiotic bacteria exert their effects in a synergistic manner to curtail and retard the growth of pathogenic and detrimental bacteria of the gut (Marteau, P R et al. Am J Clin Nutr February; 73(2 Suppl): 430S-436S; Cummings J H, et al. Am J Clin Nutr February 2001; 73(2 Suppl): 415S-420S).

It is believed that the health and well being of people can be positively or negatively influenced by the microorganisms which inhabit the gastrointestinal tract, and in particular the large bowel. These microorganisms through the production of toxins, metabolic by-products, short chain fatty acids, and the like affect the physiological condition of the host. The composition and quantity of the gut microflora can be influenced by several metabolic and physiological disorders and by conditions such as, but not limited to, diet, chemotherapy treatments, diabetes, metabolic syndromes and renal insufficiencies. If microorganisms which positively affect the health and well being of the individual can be encouraged to populate the large bowel, the physiological well being of the host is improved.

The present invention provides compositions for augmenting or maintaining kidney function in subjects including humans and animals.

### SUMMARY OF THE INVENTION

The present invention provides a composition for augmenting kidney function in a subject comprising at least one probiotic bacteria wherein said probiotic bacteria reduces creatinine and BUN levels in the subject. The composition may gelatin coating of a single or multiple layers.

The present invention further provides a composition for treatment of renal failure comprising a selected bacteria which converts nitrogenous waste into non-toxic compounds in vivo.

The present invention provides a method of inhibiting build up of toxins and metabolic wastes and overgrowth of undesirable bacteria in a subject comprising administering to a subject the composition for augmenting kidney function in the subject comprising at least one probiotic bacteria wherein said probiotic bacteria reduces creatinine and BUN levels in the subject.

The present invention provides a method of restoring and maintaining gastrointestinal health comprising administering to a subject an effective amount of the composition for augmenting kidney function in the subject comprising at least one probiotic bacteria wherein said probiotic bacteria reduces creatinine and BUN levels in the subject.

The present invention provides a method to delay or eliminate the progression of metabolic diseases comprising administering to a subject a composition for augmenting kidney function in the subject comprising at least one probiotic bacteria wherein said probiotic bacteria reduces creatinine and BUN levels in the subject.

### DETAILED DESCRIPTION OF THE INVENTION

In kidney failure there is a decrease in the glomerular filtration rate and the kidneys are unable to maintain homeostasis of the blood. Homeostatic balance of water, sodium, potassium, calcium and other salts is no longer possible and nitrogenous wastes are not excreted. Retention of water causes edema and as the concentration of hydrogen ions increases, acidosis develops. Nitrogenous wastes accumulate and a condition referred to as uremia develops in the blood and tissue. Uremic toxins can be defined as solutes that: (i) are normally excreted by healthy kidneys, (ii) accumulate progressively during the development of renal failure so that their concentration increases, and (iii) inhibit various physiologic and biochemical functions; as a whole, they contribute to a complex set of clinical symptoms that comprise the uremic syndrome. Examples of uremic toxins include, but are not limited to, ammonia, urea, creatinine, phenols, indoles, and middle molecular weight molecules. Urea levels are expressed as blood urea nitrogen readings. More specifically, in uremia, the concentration of serum creatinine, blood urea nitrogen (BUN), uric acid, and guanidino compounds such as N-methyl guanidine (NMG) and guanidino succinic acid, (GSA) are significantly altered with accompanying abnormalities in acid-base equilibrium, electrolytes and water retention. Urea is a chemical that the liver makes from ammonia. Ammonia is absorbed in the gut from the breakdown of dietary protein. After production in the liver, urea is excreted by the kidneys. Urea increases in other disease states as well including dehydration and urethral obstruction. The creatinine is a breakdown product of muscle and is excreted by the kidney at a constant rate. Creatinine serves as an important marker for kidney function. In renal failure the phosphorous levels in a subject may increase and lead to mineralization of various sites in the body. In addition there are several known and unknown substances of low and middle molecular weight which have been identified as uremic toxins which also accumulate. If untreated, acidosis and uremia can cause coma and eventually death.

The introduction of renal dialysis has contributed to rapid progress in the clinical treatment of renal failure and elucidation of uremia. When a patient has mild kidney failure where the serum creatinine level exceeds the normal range of 1.2 mg/dl or less, the patient does not require renal replacement therapy such as dialysis or renal transplant. However, in general, when the serum creatinine level rises to 13.6±4.6 mg/dl., the patient needs routine dialysis or a kidney transplant to survive.

Dialysis can serve as a lifetime therapy for ESRD patients. Phosphate binders such as Renagel® (sold by Geltex/Genzyme, Boston, Mass.), calcium acetate, calcium carbonate or aluminum hydroxide are generally prescribed for uremic patients receiving dialysis to reduce elevated phosphate levels. In general, however, dialysis is very expensive, inconvenient, time consuming and may occasionally produce one or more side effects. With a successful kidney transplant, a patient can live a more normal life with less long-term expense. However, there are also high costs associated with transplant surgery, the recovery period and the continuous need for anti-rejection medications. Further, there are often times a shortage of suitable donors. Accordingly, there is a need for alternative strategies.

The present invention provides a composition for augmenting kidney function in a subject comprising at least one probiotic bacteria wherein said probiotic bacteria reduces creatinine and BUN levels in the subject. The composition may comprise a coating or encapsulation in a gel capsule so that the composition may reach the intestines of the subject when ingested orally. One such coating is a double layered gelatin coating. However, the present invention may comprise additional marketable forms such as a nutriceuticals, dietary supplements and powders, health bars, yogurts, tablets, dry foods, and pet formulations.

The probiotic bacteria useful in the present invention include: Lactobacillus acidophilus, L. bulgaricus, L. casei, L. rhamnosus, L. fermentum, L. salivaroes, L. brevis, L. plantarum, L. ruteri, S. thermophilus, Bacillus sporogenes, Bifidobacterium adolescentis, B. infantis, B. longum , B. thermophilus, B. pasteurii and L. sporogenes or B. bifidum. In a preferred embodiment the probiotic bacteria comprises one or more of the following: L. bulgaricus, S. thermophilus, L. acidophilus or B. bifidum, see Table 1 below.

It has been found that the probiotic bacteria are more effective in ranges of from 10⁹ to 10¹¹ cfu. For instance, Lactobacillus acidophilus is of particular use between 10⁹ to 10¹⁰ cfu, whereas B. longum is preferred for use between 10⁹ to 10¹⁰ cfu, and S. thermophilus between 10¹⁰ to 10¹¹ cfu. One skilled in the art can routinely determine the appropriate ranges of the probiotic bacteria based upon desired application and use.

The composition of the present invention may further comprise at least one vitamin component and at least one mineral component. In this aspect the composition may take the form of an enhanced multi-vitamin or calcium supplement. The composition of the present invention is administered to a subject to alleviate the symptoms of uremia associated with toxic metabolites flowing into the bowel of patients taking cancer therapy drugs. Further, the composition of the present invention is useful when administered to a subject to alleviate the increased creatinine or urea levels in subjects undergoing HIV or AIDS chemotherapy treatment; suffering from metabolic syndrome; or for subjects consuming high protein and low carbohydrate diets. Metabolic syndrome or metabolic disease is regarded as present in patients with borderline symptoms of diseases including diabetes and hypertension. The composition of the present invention can be used to delay or prevent the progression of the metabolic disease to its advanced status resulting in diabetes, hypertension or other diseases.

The present invention provides a composition comprising one or more selected bacteria which when instilled into the gastrointestinal tract of a subject converts nitrogenous wastes accumulated in the subject due to renal insufficiency into nontoxic compounds. The term subject is meant to include humans and animals, including pigs, horses, cats and dogs. In subjects with renal failure, higher concentrations of nitrogenous solutes traverse intestinal capillaries into the bowel of the subjects through diffusion. The composition of the present invention can be administered orally, or through any other appropriate manner so that the selected bacteria are instilled into the gastrointestinal tract of the subject and nitrogenous wastes are reduced. The selected bacteria are live bacteria which consume excess urea, creatinine, and “uremic” solutes, including for example S. thermophilus, L. bulgaricus, L. acidophilus, and B. Bifidus.

The present invention further provides a method of inhibiting build up of toxins and metabolic wastes and overgrowth of undesirable bacteria in a subject comprising administering to a subject the composition comprising at least one probiotic bacteria wherein said probiotic bacteria reduces creatinine and BUN levels in the subject.

The present invention further provides a method of restoring and maintaining gastrointestinal health comprising administering to a subject an effective amount of the composition comprising at least one probiotic bacteria wherein said probiotic bacteria reduces creatinine and BUN levels in the subject.

The present invention further provides an in vivo method of reducing nitrogenous wastes or toxins due to renal failure in a subject comprising instilling selected bacteria into the gastrointestinal tract of the subject so that nitrogenous wastes are converted into nontoxic compounds by the selected bacteria. When used in such a method the composition effectively converts nitrogenous waste into additional bacteria growth or non-toxic compounds in vivo, which are then excreted in the feces. The selected bacteria of the compositions of the present invention may be instilled via any suitable means including but not limited oral administration of the selected bacteria as a pharmaceutical composition or food stuff, injection, surgical implantation, or intranasal administration. It is preferred that the compositions of the present invention be administered to the subject on a routine basis such as one or more times daily over a period of time. Reduction of nitrogenous wastes is indicated via blood, urine or fecal sample testing wherein a stabilization or reduction in BUN levels or serum creatinine levels of the blood, urine or fecal samples as compared to initial or control levels indicates effective treatment. The compositions of the present invention are useful for treatment of renal failure. The compositions can be administered to the subject to alleviate the symptoms of uremia caused by chemotherapeutic drug programs, diabetic kidney related failures, kidney disease or an inborn error of urea metabolism. The compositions of the present invention may be administered to treat renal insufficiency, liver insufficiency, inborn error of urea metabolism or gastrointestinal disorders and diseases.

The present invention further provides compositions for treating uremia or renal dysfunction in a subject, comprising a mixture of one or more selected probiotic bacteria which converts nitrogenous waste into non-toxic compounds in vivo along with one or more of the following: a prebiotic, ammoniaphilic bacteria with high urease activity, and/or sorbents with specific adsorption affinities for uremic toxins such as creatinine, uric acid, phenols, indoles, middle molecular weight molecules and inorganic phosphate along with a water sorbent, for use in the alleviation of uremia. A prebiotic is a non-digestible food ingredient that beneficially affects the host by selectively stimulating the growth and/or the activity of one or a limited number of bacteria in the colon. Prebiotics are typically thought of as carbohydrates of relatively short chain length.

Compositions comprising one or more of the selected probiotic bacteria which converts nitrogenous waste into non-toxic compounds in vivo may be enteric coated or microencapsulated. i.e., in a gel cap or other desired compositions. Enteric coating of the composition is specifically designed to deliver sorbents and/or the selected bacterial source at the ileal and colonic regions of the bowel where maximal resorption of uremic solutes and other molecules are found to occur. This is preferably achieved via a coating material that disintegrates and dissolves at a neutral pH, i.e., pH of 6.6 to 7.5 or higher. Examples of enteric coatings with these characteristics include, but are not limited to, Zein, alginic acid, polyglycolactic acid, polylactic acid, polylactide-co-glycolide and similar coating materials. Enteric coatings also enable delivery of the sorbents to their site of action in relatively native form without binding of various digestive materials to the sorbents prior to reaching the target region.

Compositions of the present invention may further comprise a phosphate binding agent such as aluminum hydroxide gel, calcium carbonate or calcium acetate, magnesium hydroxide gel and/or a water binding agent such as psyllium fibers, naturally occurring gums such as locust bean gum, guar gum or modified starches.

Compositions of the present invention are administered orally to subjects in need thereof to decrease the build-up of toxins and metabolic wastes and/or to inhibit or decrease the over growth of undesirable bacteria in the subject. In one embodiment, the composition is administered to a subject with uremia to alleviate the symptoms of uremia. By “alleviation of symptoms” of uremia, it is meant that the composition removes sufficient levels of uremic toxins such that a patient suffering from uremia either does not require dialysis, requires dialysis less frequently or for shorter durations, or does not require initiation of dialysis as soon as would be needed without treatment. Compositions of the present invention can also be administered to a subject in need thereof to treat not only renal insufficiency and inborn error of urea metabolism, but also liver insufficiency and gastrointestinal disorders and diseases.

The delivery of the compositions of the present invention may be via pharmaceutical compositions of liquid, capsule, pill or other suitable forms. An effective amount of probiotic or selected bacteria of this invention can be administered along with a mixture of sorbents in the emulsion or paste or separately in an ingestible capsule. An “effective amount” as used herein is an amount necessary to achieve a selected result. For example, an effective amount of a bacteria and fiber-containing composition useful for reducing pathogenic microorganisms in the gastrointestinal tract is an amount that achieves the selected result of reducing the pathogenic microorganisms. Such an amount is readily determined without undue experimentation by those skilled in art.

Toxic nitrogenous waste products can accumulate in the intestinal tract of a subject when the normal balance of intestinal microbes is disrupted. Nitrogenous waste products include, but are not limited to, urea, uric acid, creatinine, ammonia, etc. These products will also tend to accumulate in the gastrointestinal tract in any condition which disrupts the kidneys ability to excrete the build up of nitrogenous waste products in the blood, thereby resulting in the diffusion of the nitrogenous waste products from the circulating blood into the bowel. Exemplary conditions that affect nitrogen metabolism include, but are not limited to, high protein consumption, chemotherapy, metabolic diseases, defects in protein metabolism, and nucleic acid metabolism. Diabetes, kidney failure, and liver disease, as well as other conditions can result in the build up of toxic nitrogenous compounds in the blood such as specifically chemotherapy for cancer, HIV, and AIDS.

The probiotic formulation in the compositions and provided herein will benefit the overall general condition of the gastrointestinal tract. This is especially beneficial to subjects with a propensity to gastrointestinal complaints, such as those undergoing chemotherapy, eating high protein diets, and subjects with metabolic syndromes. The precise amount of the compositions of the present invention ingested by a subject may be decided according to the judgment of a practitioner or dietician and each subject's circumstances. The present invention is further illustrated by the following non-limiting examples.

### EXAMPLES

### Example 1

### Bun/Creatinine Decrease

Different formulations were tested on ⅚th nephrectomized mini pigs. From a total of 20 pigs obtained over a six month period, three died post-surgery (one of the three had to be euthanized due to extreme sickness) and two more were euthanized due to acute illness, possibly due to infection.

The mini pigs that were fed formulation 1 were subsequently separated and used again for feeding formulations 2A and 2B. Likewise, 2A was reused for administering formulation 2Ac. A switch was performed using the following procedure. The initial bacterial regimen was stopped and a wash out period of 3 to 4 weeks was allowed prior to a switch to a new treatment regimen. Mini pigs in the other groups were subjected exclusively to the assigned food regimen (3A, 3B, 4A, 5A, respectively). All of the screened, chosen and specially employed probiotic microbial strains are Generally recognized as safe (GRAS) classification by FDA except those obtained in the formulations if B. pastuerii.

Weight measurement, blood draw and analysis were performed at different time intervals. Due to the small sample size, as well as difference in body weights and the measurement intervals, regression analysis and curve fitting were used to assess the data. Generally, these mathematical techniques are used to explain and/or predict additional data points. Present analysis determined changes in body weight, BUN, and creatinine levels for each mini pig. The details of the formula, number of pigs, delivery mode, daily dosage, duration, composition, and a general summary of the findings are listed in Table 1.

TABLE 1

| Formula (# of | Delivery mode/daily | | |
|---|---|---|---|
| pigs) | dosage/duration (days) | Composition per dose, CFU | Summary of Findings |
| 1 (6) | Double layered gelatin | S. thermophilus, L. bulgaricus, | BUN decrease |
| | capsule/1 to 12/27 to | L. acidophilus, B. bifidus (10 × 10⁹/cap) | Creatinine slight decrease |
| | 50 | (1:1:1:1) | Weight increase |
| 2A (2) | Frozen food ball/1 to 2/ | B. pasteurii (5 × 10⁹), B. coagulans | BUN increase |
| | 69 | (10 × 10⁹) | Creatinine slight decrease |
| | | | Weight increase |
| 2B (3) | Frozen food ball/1 to 2/ | B. coagulans (10 × 10⁹) | BUN slight decrease |
| | 69 | | Creatinine slight decrease |
| | | | Weight stable |
| 2Ac (2) | Tablet/10/15 | B. coagulans (0.78 × 10⁸/tab) | BUN increase |
| | | | Creatinine slight decrease |
| | | | Weight slight decrease |
| 3A (3) | Frozen food balls/1 to 4/ | S. thermophilus (11.6 × 10⁹), | BUN stable |
| | 100 | L. acidophilus (1 × 10⁹), B. longum | Creatinine stable |
| | | (1 × 10⁹) | Weight stable |
| 3B (2) | Frozen food balls/1 to 4/ | L. acidophilus (1 × 10⁹), B. longum | BUN stable |
| | 100 | (1 × 10⁹) | Creatinine stable |
| | | | Weight stable |
| 4A (3) | Frozen food balls/1 to | S. thermophilus (11.6 × 10⁹), L. acidophilus | BUN stable |
| | 10/51 | (1 × 10⁹), B. longum | Creatinine slight decrease |
| | | (1 × 10⁹) | Weight increase |
| 5A (2) | Gelatin capsule/3 to 10/ | S. thermophilus (20.4 × 10⁹), | BUN decrease |
| | 20 (ongoing) | L. acidophilus (1 × 10⁹), B. longum | Creatinine decrease |
| | | (1 × 10⁹) | Weight inconclusive |

Of the several probiotic oral formulations tested, a specific formulation of four microbial strains (Formulation 1) on a low frequency dosage regimen in these ⅚th Nephrectomized Mini pigs (n=6) exhibited (a) continued gain in body weights-approximately 33%, (b) decreased BUN and as well creatinine levels decreased by approximately 13%. In a similar manner two mini pigs on formulation 5A also demonstrated decreased BUN and Creatinine levels, although the body weight of one mini pig increased and that of the other decreased. In addition, the formulations 3A, 3b and 4a demonstrates stable or slight increase in body weights, somewhat stable or slight decreased levels of urea and creatinine levels. In general, all of these data and findings reflect that at least nitrogenous waste metabolites (urea and creatinine) were not accumulating in the blood. These results show that a suitable combination of selectively chosen probiotic microbes are suitable for oral gut-based uremia therapy.

### Example 2

### Sprague-Daly Rats

For example, ⅚th nephrectomized sprague-Daly rats weighing 281.20=/−41. 6 gm were subjected to oral ingestion of various probiotic formulations. Measurement of baseline weight, BUN, serum creatinine, urine volume and fecal flora composition were determined. The study group consisted of 36 nephrectomized rats and 6 normal rats as controls. After a two-week post surgery stabilization, cohorts of six groups of rats were fed standard rat chow plus one of the following regimens: 1) placebo, 2) B. pasteurii 3) L. sporogenes, 4) L. acidophilus, L. bulgaricus; Bifidus, S. thermophilus, L. casei, and L. reuteri, 5) L. acidophilus, L. bulgaricus, Bifidus, S. thermophilus and 6) S. boulardii. Subsequent blood, urine, and fecal measurements were obtained every 30 days for a total of 120 days.

The subtotally nephrectomized rats fed B. pasteurii and L. sporogenes had lower BUN levels (62.0±21, 63.0±26 mg/dl) compared with placebo (99.0±46 mg/dl) a reduction of (38 and 37%). Serum creatinine levels were similarly reduced in rats fed with B. pasteurii and L. sporogenes (0.9±).25, 0.9±0.2 mg/dl) compared to placebo (1.5±0.56 mg/dl) a reduction of 40% in both groups. Rats fed with regimens comprising only L. acidophilus, L. bulgaricus; Bifidus, S. thermophilus, L. casei, L. reuteri, L. acidophilus, L. bulgaricus, Bifidus, S. thermophilus or S. boulardii did not show significant difference in BUN or serum creatinine, compared to placebo. Feeding increased the fecal count for the appropriate group of bacteria in all groups at eight weeks. These results indicate that B. pasteurii and L. sporogenes administered orally as dietary supplements metabolize urea and creatinine in vivo in subjects. Whether similar activity is discerned in uremic patients and large animals is the subject of derivative study. L. acidophilus (NCFM) fed dialysis patients reduced uremic toxins and showed improved nutritional status of about a ten percent increase in daily caloric intake and 1.6% increase in BMI (p<0.05) with no side effects. This study used a rat model of CRF (5/6 nephrectomy) to test 6 non-pathogenic microorganisms (MO) for possible use in a probiotic product. Sixty rats had ⅚ nephrectomies performed. Baseline creatinine levels(Scr), BUN were measured and Cr clearance calculated. Rats (18 male and 18 female) with sufficient renal impairment (Scr=1.0=/−0.4) were distributed into 6 matched groups (GP), ANOVA showed no significant difference between groups (p=0.516) at baseline. Rats were individually caged and fed a special diet beginning at day 30 supplemented with a particular MO additive daily for up to 126 days. Periodic BW, Scr, BUN and CrCl were measured. A control group of non-nephrectomized rats(n=7, Scr=0.2±0.1) received the same food without any supplement. All of the rats survived (Scr at end=0.5=/−0.1). Days of survival was the primary endpoint variable. The study ended at day 156.

TABLE 2
Survival in Groups of Rats Receiving Oral Diet
Supplements Containing Non-Pathogenic Microorganism

| | | | | % | Mean | | |
|---|---|---|---|---|---|---|---|
| GP | Organism | ALIVE | DEAD | SURVIVE | days | SD | Median |
| G | S.boulardi | 2 | 4 | 33.3 | 111 | 44 | 113 |
| B | Placebo | 2 | 4 | 33.3 | 116 | 39 | 122 |
| F | H1001 | 2 | 4 | 33.3 | 116 | 36 | 110 |
| E | SF101 | 3 | 3 | 50 | 126 | 33 | 132 |
| C | B. pasteruii | 4 | 2 | 66.7 | 148 | 14 | 156 |
| D | L. sporogenes | 5 | 1 | 83.3 | 149 | 16 | 156 |

As shown in Table 2, diets D and C were more effective than G, B, and F (p<0.05). The study showed that a probiotic containing either or both B. pasteurii and L. sporogenes is capable of increasing survival in otherwise untreated uremic rats.

What is claimed is:

1. A composition for augmenting kidney function in a subject comprising at least one probiotic bacteria wherein said probiotic bacteria reduces creatinine and BUN levels in the subject.

2. The composition of claim 1 further comprising a gelatin coating.

3. The composition of claim 1 wherein said at least one probiotic bacteria is Lactobacillus acidophilus, L. bulgaricus, L. casei, L. rhamnosus, L. fermentum, L. salivaroes, L. brevis, L. plantarum, L. ruteri, B. pasteurii, S. thermophilus, Bacillus sporogenes, Bifidobacterium adolescentis, B. infantis, B. longum, B. thermophilus or B. bifidum.

4. The composition of claim 1 further comprising at least one vitamin component and at least one mineral component.

5. A composition for treatment of renal failure comprising a selected bacteria which converts nitrogenous waste into non-toxic compounds in vivo.

6. A method of inhibiting build up of toxins and metabolic wastes and overgrowth of undesirable bacteria in a subject comprising administering to a subject the composition of claim 1.

7. The method of claim 6 wherein the composition is administered to a subject to alleviate the symptoms of uremia associated with toxic metabolites flowing into the bowel of patients afflicted with cancer due to cancer therapy drugs.

8. The method of claim 6 wherein the composition is administered to a subject to alleviate the increased creatinine or BUN levels in subjects undergoing HIV or AIDS chemotherapy treatment.

9. The method of claim 6 wherein the composition is administered to a subject to alleviate the increased creatinine or BUN levels in subjects with metabolic syndrome.

10. The method of claim 6 wherein the composition is administered to a subject to alleviate the symptoms of increased creatinine or BUN levels in subjects consuming high protein and low carbohydrate diets.

11. A method of restoring and maintaining gastrointestinal health comprising administering to a subject an effective amount of the composition of claim 1.

12. A method of to delay or eliminate the progression of metabolic diseases comprising administering to a subject a composition for augmenting kidney function in the subject comprising at least one probiotic bacteria wherein said probiotic bacteria reduces creatinine and BUN levels in the subject.

13. The composition of claim 1 wherein the probiotic bacteria comprise Lactobacillus acidophilus, S. thermophilus, and B. longum.
